(19) United States
(12) Patent Application Publication
(10) Pub. No.: US 2020/0087168 A1
(43) Pub. Date: Mar. 19, 2020

(54) CARBON NANOFIBER COMPOSITE WATER PURIFICATION MATERIAL, PREPARATION METHOD THEREOF, AND APPLICATION THEREOF

(71) Applicant: GUANGZHOU LANQUAN HOUSEHOLD ELECTRIC APPLIANCE CO., LTD., Guangzhou (CN)

(72) Inventor: Zuoguo Cao, Guangzhou (CN)

(21) Appl. No.: 16/417,654

(22) Filed: May 21, 2019

(30) Foreign Application Priority Data
Sep. 19, 2018 (CN) 201811093669.4

(51) Int. Cl. C02F 1/28; B01J 20/28; B01J 20/20; B01J 20/24; A61K 8/9789; A61Q 19/02; A61Q 19/00

(52) U.S. Cl. CPC C02F 1/28; B01J 20/2805; B01J 20/205; B01J 20/24; C02F 2101/20; A61K 8/9789; A61Q 19/02; A61Q 19/007; B01J 20/28023

(57) ABSTRACT

The present invention involves a carbon nanofiber composite water purification material. The carbon nanofiber composite water purification material is prepared by the following components in parts by weight: 70-80 parts of activated carbon cellulose, 6-10 parts of lemon powder, 6-10 parts of Jew's ear powder, and 1-2 parts of graphene oxide. The present invention also involves a filter cartridge. The filter cartridge includes a filter cartridge body comprising a plastic skeleton and a flat membrane which coats an outer surface of the plastic skeleton; the plastic skeleton is provided with a plurality of water passage holes; the carbon nanofiber composite water purification material is provided on an outer surface of the flat membrane. The carbon nanofiber composite waste purification material of the present invention has high nano-porosity, strong adsorption abilities, and a long lifetime.

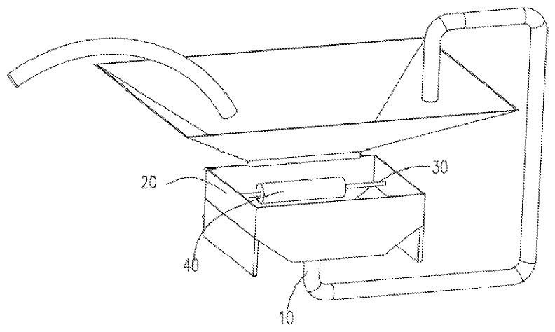

### CROSS-REFERENCE TO RELATED APPLICATIONS

The present application claims the benefit of Chinese Patent Application No. 201811093669.4 filed on Sep. 19, 2018. The contents of the above are hereby incorporated by reference.

### FIELD OF THE INVENTION

The invention relates to a water purification material, in particular to a carbon nanofiber composite water purification material, a preparation method thereof, and an application thereof.

### BACKGROUND OF THE INVENTION

With the rapid development of science and technology, an increasing amount of industrial and domestic wastewater is discharged, which leads to serious environmental pollution and severely threatens human health. There is a pressing need to eliminate heavy metal hazards and to recover heavy metals from wastewater. Among the many methods of treating metal ions in wastewater, the adsorption method is one of the best. Using adsorbents to remove metal ions from wastewater has a number of advantages including effective, low in cost, and has wide applicability; in addition, useful metals can be recovered, no secondary pollution is produced, and adsorbents can be reused. Among the common adsorbents, activated carbon has good metal adsorbing ability; however, its regeneration is complicated. Consequently, in recent years, cellulose has been receiving attention as a biodegradable and non-polluting renewable resource.

Chinese invention patent No. CN 201610783089.2 has disclosed a carbon fiber composite material and its application. The carbon fiber composite material is prepared from the following components in percentage by weight: 30-40 parts of activated carbon, 30-40 parts of activated carbon fiber, 10-15 parts of a synthetic fiber, 8-15 parts of calcium sulfite powder, 5-10 parts of zinc alloy powder, 0.5-2 parts of a binding agent, and 90-100 parts of water. The calcium sulfite powder and copper-zinc alloy powder are dispersed into the pores of the carbon fiber composite material, increasing the contact area between the carbon fiber composite material and the water being filtered, prolonging the filtering process and allowing thorough filtration. As a result, compared to traditional carbon fiber materials, the carbon fiber composite material of this invention is more capable of removing chlorine from a large flow of water. However, the adsorption ability of this carbon fiber composite material still has room for improvement. In addition, it is only able to carry out one function; it lacks other useful features including scale inhibition, sterilization, and oxidation resistance.

### SUMMARY OF THE INVENTION

To overcome the deficiencies of the prior art, the first objective of the present invention is to provide a carbon nanofiber composite waste purification material that has high nano-porosity, strong adsorption abilities, and a long lifetime; in addition, it is able to filter out heavy metals, prevent scale formation and has sterilization and oxidation resistance abilities.

The second objective of the present invention is to provide a preparation method of the carbon nanofiber composite waste purification material.

The third objective of the present invention is to provide a filter cartridge that contains the carbon nanofiber composite waste purification material described above.

The fourth objective of the present invention is to provide a method of manufacturing the filter cartridge.

The first objective of the present invention can be achieved by the following technical solution:

A carbon nanofiber composite water purification material, which is prepared by the following components in parts by weight: 70-80 parts of activated carbon cellulose, 6-10 parts of lemon powder, 6-10 parts of Jew's ear (Auricularia auricula-judae) powder, and 1-2 parts of graphene oxide.

The second objective of the present invention can be achieved by the following technical solution:

A preparation method of the carbon nanofiber composite water purification material, comprising the following steps:

Step A—crushing and sieving: grinding the activated carbon cellulose, the lemon powder, the Jew's ear powder, and the graphene oxide respectively; sieving through a 100-200 mesh filter respectively; and

Step B—mixing: mixing a predetermined amount of the activated carbon cellulose, the lemon powder, the Jew's ear powder, and the graphene oxide to obtain the carbon nanofiber composite water purification material.

The third objective of the present invention can be achieved by the following technical solution:

A filter cartridge, which includes a filter cartridge body, the filter cartridge body comprises a plastic skeleton and a flat membrane which coats an outer surface of the plastic skeleton; the plastic skeleton is provided with a plurality of water passage holes; the carbon nanofiber composite water purification material according to the present invention is provided on an outer surface of the flat membrane.

Preferably, the carbon nanofiber composite water purification material is attached to the outer surface of the flat membrane by wet centrifugal molding.

Preferably, a nonwoven fabric which coats the filter cartridge is provided.

Preferably, the plastic skeleton is cylindrically-shaped; the flat membrane is ring-shaped; the flat membrane is coated on the plastic skeleton by welding; the flat membrane has a diameter of 0.1-0.3 μm.

The fourth objective of the present invention can be achieved by the following technical solution:

A method of manufacturing the filter cartridge, wherein it comprises the following steps:

Step I—slurry preparation: mixing the carbon nanofiber composite water purification material with water thoroughly to obtain a slurry, wherein the amount of the water added is 50% to 60% of a total weight of the carbon nanofiber composite water purification material;

Step II—filter cartridge body preparation: providing the plastic skeleton and the flat membrane, coating the flat membrane on the outer surface of the plastic skeleton to obtain the filter cartridge body;

Step III—wet centrifugal molding: providing a wet centrifugal molding device, which comprises a vacuuming device, a molding chamber, a positioning frame provided in the molding chamber; the positioning frame is provided with a rotating device; communicating a vacuum port of the vacuuming device with an inner chamber of the molding chamber via a pipe; installing the filter cartridge body on the rotating device; pumping the slurry into the molding chamber via the vacuuming device, and centrifuging the filter cartridge body at a high speed by the rotating device simultaneously; under a dual action of vacuum adsorption and centrifugation, the slurry uniformly binds to the outer surface of the flat membrane of the filter cartridge body to obtain a semifinished filter cartridge; and

Step IV—water removal: centrifuging and drying the semifinished filter cartridge obtained in step III sequentially to completely remove water in the semifinished filter cartridge in order to obtain the filter cartridge.

Preferably, after completing step IV, it is also required to coat a nonwoven fabric on the outer surface of the filter cartridge.

Preferably, in step III, the rotating device operates at a rotational speed of 5000 rpm for 30-90 seconds.

Preferably, in step IV, centrifugation is carried out at a speed of 500 rpm for 20-50 seconds; drying is carried out at a temperature of 105-120° C. for 4-6 hours.

The advantageous effects of the present invention are as follows:

The carbon nanofiber composite water purification material of the present invention is prepared by food-grade activated carbon cellulose, food-grade lemon powder, food-grade Jew's ear powder, and food-grade graphene oxide.

The food-grade activated carbon cellulose has developed pores and could effectively adsorb a number of substances including organic matter, pesticide residues, antibiotics, and heavy metals from water, which improves the taste of water. A filter cartridge containing the activated carbon cellulose has the following advantages: it is light in weight, has a fast filtering speed, good filtering abilities, and a long lifetime. The filter cartridge allows the development of water purifiers which are lighter and smaller.

The food-grade lemon powder contains a lot of vitamin C from lemon; vitamin C could effectively inhibit the activity of leucine and could reduce the oxidative melanin to its colorless reduced form, which aids the whitening of skin and the removal of freckles. Lemon also contains a large amount of citric acid, which promotes skin exfoliation and the regeneration of epidermal cells, maintains skin moisture, improves skin texture and restores skin elasticity. A mixture of lemon powder and the activated carbon cellulose has anti-oxidation properties; a bath water purifier containing such a mixture potentially has a skin protective effect.

The food-grade Jew's ear powder is made by a number of processes including soaking, disinfecting, rough selection, washing, dehydrating, drying, and grinding. The powder has high nutritional and medicinal value. Jew's ear is a fungus that is rich in plant collagen and strongly adsorbs of calcium and magnesium ions; it forms coordination compounds with calcium and magnesium ions to prevent the formation of calcium carbonate and magnesium carbonate. Hence, Jew's ear is able to effectively reduce the hardness of water and prevent the formation of scale. A filter that contains Jew's ear powder added to the activated carbon cellulose is capable of inhibiting the formation of scale.

When the food-grade graphene oxide is in direct contact with bacteria in water, it acts like a nano-scaled blade and directly destroys the structure of bacterial membranes mechanically. In addition, as graphene oxide has excellent electron transport abilities, it is able to easily change the membrane potential of the bacterial membrane, disturbing its equilibrium. As a result, cellular respiration, electron transportation, and signal transduction at the membrane are disturbed, resulting in the death of the bacteria. A filter that contains graphene oxide added to the activated carbon cellulose is capable of sterilization; its sterilization rate is over 99%.

In view of the above, the carbon nanofiber composite waste purification material of the present invention has high nano-porosity, strong adsorption abilities, and a long lifetime; in addition, it is able to filter out heavy metals and prevent scale formation, and has sterilization and oxidation resistance abilities.

### BRIEF DESCRIPTION OF THE DRAWINGS

The sole FIGURE is a schematic diagram showing the wet centrifugal molding device in embodiment 1.

### REFERENCE NUMERALS

10—vacuuming device; 20—molding chamber; 30—positioning frame; 40—filter cartridge body.

### DETAILED DESCRIPTION OF THE ILLUSTRATED EMBODIMENTS

The present invention is further described in combination with the illustrated embodiments below. It should be noted that a new embodiment may be obtained by a combination of the embodiments or any of the technical features described below, provided that the technical features do not contradict with each other.

A carbon nanofiber composite water purification material, which is prepared by the following components in parts by weight: 70-80 parts of activated carbon cellulose, 6-10 parts of lemon powder, 6-10 parts of Jew's ear (Auricularia auricula-judae) powder, and 1-2 parts of graphene oxide.

A preparation method of the carbon nanofiber composite water purification material, comprising the following steps:

Step A—crushing and sieving: grinding activated carbon cellulose, lemon powder, Jew's ear powder, and graphene oxide respectively; sieving the ground substances through a 100-200 mesh filter respectively; and

Step B—mixing: mixing a predetermined amount of the activated carbon cellulose, lemon powder, Jew's ear powder, and graphene oxide from the previous step to obtain the carbon nanofiber composite water purification material.

A filter cartridge, which includes a filter cartridge body, the filter cartridge body comprises a plastic skeleton and a flat membrane which coats an outer surface of the plastic skeleton; the plastic skeleton is provided with a plurality of water passage holes; the carbon nanofiber composite water purification material according to the present invention is provided on an outer surface of the flat membrane.

As a further embodiment of the present invention, the carbon nanofiber composite water purification material binds to the outer surface of the flat membrane by wet centrifugal molding.

The carbon nanofiber composite water purifying material of the present invention does not comprise a binding agent. It is well known that the main function of the binding agent is to bind together materials of different properties; however, the adhesive may also disperse to the holes of carbon fiber materials, blocking these holes. This seriously affects the filtration ability of carbon fiber filters. The invention adopts wet centrifugal molding and hence does not require a binding agent.

As a further embodiment of the present invention, a nonwoven fabric which coats the filter cartridge is provided.

As a further embodiment of the present invention, the plastic skeleton is cylindrically-shaped; the flat membrane is ring-shaped; the flat membrane is coated on the plastic skeleton by welding; the flat membrane has a diameter of 0.1-0.3 μm.

A method of manufacturing a filter cartridge, comprising the following steps:

Step I—slurry preparation: mixing the carbon nanofiber composite water purification material with water thoroughly to obtain a slurry; the amount of the water added is 60% of the total weight of the carbon nanofiber composite water purification material.

Step II—filter cartridge body preparation: providing a plastic skeleton and a flat membrane, coating the flat membrane on the outer surface of the plastic skeleton to obtain the filter cartridge body; the filter cartridge body is mainly used for shaping the filter cartridge, its inner hole forms a water channel. When drying the filter cartridge, an inner lining is provided to prevent deformation of the filter cartridge.

Step III—wet centrifugal molding: providing a wet centrifugal molding device, which comprises a vacuuming device, a molding chamber, a positioning frame provided in the molding chamber; the positioning frame is provided with a rotating device; communicating the vacuum port of the vacuuming device with the inner chamber of the molding chamber via a pipe; installing the filter cartridge body on the rotating device; pumping the slurry into the molding chamber via the vacuuming device, and centrifuging the filter cartridge body at a high speed by the rotating device simultaneously. Under the dual action of vacuum adsorption and centrifugation, the slurry uniformly binds to the outer surface of the flat membrane of the filter cartridge body to obtain a semifinished filter cartridge.

Step IV—water removal: centrifuging and drying the semifinished filter cartridge obtained in step III sequentially to completely remove the water in the semifinished filter cartridge and to obtain the filter cartridge. Centrifugation is carried out first, which allows the subsequent drying process to be shortened; this greatly increases efficiency, productivity, and reduces energy consumption.

As a further embodiment of the present invention, after step IV is completed, it is also required to coat a nonwoven fabric on the outer surface of the filter cartridge. The non-woven fabric acts to prevent the occurrence of black water when using the filter cartridge.

As a further embodiment of the present invention, in step III, the rotating device operates at a rotational speed of 5000 rpm for 30-90 seconds.

As a further embodiment of the present invention, in step IV, centrifugation is carried out at a speed of 500 rpm for 20-50 seconds; drying is carried out at a temperature of 105-120° C. for 4-6 hours.

Below are specific examples of the present invention. The raw materials, equipment, and the like employed in the following examples can be obtained commercially unless otherwise specified.

### Embodiment 1

A carbon nanofiber composite water purification material, which is prepared by the following components in parts by weight: 75 parts of activated carbon cellulose, 8 parts of lemon powder, 8 parts of Jew's ear powder, and 1.5 parts of graphene oxide.

The preparation method of the carbon nanofiber composite water purification material comprises the following steps:

Step A—crushing and sieving: grinding activated carbon cellulose, lemon powder, Jew's ear powder, and graphene oxide respectively; sieving the ground substances through a 150 mesh filter respectively; and

Step B—mixing: according to the amount specified above, mixing the of the activated carbon cellulose, lemon powder, Jew's ear powder, and graphene oxide from the previous step to obtain the carbon nanofiber composite water purification material.

A method of manufacturing a filter cartridge, comprising the following steps:

Step I—slurry preparation: mixing the carbon nanofiber composite water purification material with water thoroughly to obtain a slurry; the amount of the water added is 60% of the total weight of the carbon nanofiber composite water purification material.

Step II—filter cartridge body preparation: providing a plastic skeleton and a flat membrane, coating the flat membrane on the outer surface of the plastic skeleton to obtain the filter cartridge body; the filter cartridge body is mainly used for shaping the filter cartridge, its inner hole forms a water channel. When drying the filter cartridge, an inner lining is provided to prevent deformation of the filter cartridge.

Step III—wet centrifugal molding: providing a wet centrifugal molding device (see the FIGURE), which comprises a vacuuming device 10, a molding chamber 20, a positioning frame 30 provided in the molding chamber 20; the positioning frame 30 is provided with a rotating device; communicating the vacuum port of the vacuuming device with the inner chamber of the molding chamber via a pipe; mounting the filter cartridge body 40 on the rotating device; pumping the slurry into the molding chamber via the vacuuming device, the rotating device also drives the high-speed centrifugation of the filter cartridge body. Under the dual action of vacuum adsorption and centrifugation, the slurry uniformly binds to the outer surface of the flat membrane of the filter cartridge body to obtain a semifinished filter cartridge. The rotating device operates at a rotational speed of 5000 rpm for 60 seconds.

Step IV—water removal: centrifuging and drying the semifinished filter cartridge obtained in step III sequentially to completely remove the water in the semifinished filter cartridge in order to obtain the filter cartridge. Centrifugation is carried out at a speed is 500 rpm for 20-50 seconds; drying is carried out at a temperature of 110° C. for 5 hours.

After step IV is completed, it is also required to coat a nonwoven fabric on the outer surface of the filter cartridge.

### Embodiment 2

A carbon nanofiber composite water purification material, which is prepared by the following components in parts by weight: 78 parts of activated carbon cellulose, 7 parts of lemon powder, 9 parts of Jew's ear powder, and 1 part of graphene oxide.

The rest is the same as embodiment 1.

### Embodiment 3

A carbon nanofiber composite water purification material, which is prepared by the following components in parts by weight: 70 parts of activated carbon cellulose, 6 parts of lemon powder, 6 parts of Jew's ear powder, and 1 part of graphene oxide.

The rest is the same as embodiment 1.

### Embodiment 4

A carbon nanofiber composite water purification material, which is prepared by the following components in parts by weight: 80 parts of activated carbon cellulose, 10 parts of lemon powder, 10 parts of Jew's ear powder, and 2 parts of graphene oxide.

The rest is the same as embodiment 1.

### Tests

Iodine and Methylene Blue Adsorption Measurement:

Iodine and methylene blue solutions are respectively added dropwise to 1 g of the carbon nanofiber composite water purification material. The mixtures obtained are stirred until the color disappears. At this point, the carbon nanofiber composite materials are saturated and unable to continue adsorbing iodine and methylene blue. The titration values are recorded, which correspond to adsorption values.

Residual Chlorine Adsorption Lifetime Test:

The filter cartridge made from the carbon nanofiber composite water purification material is connected to the water outlet pipe of a labeled water tank. Residual chlorine in the labeled water tank is maintained at a concentration of 1 mg/L. Water from the labeled water tank is filtered through the filter cartridge, and the concentration of residual chlorine in the filtered water is measured. It was found that in 50 tons of filtered water, the residual chlorine removal percentage exceeds 75% (according to the requirement of NSF standards, the residual chlorine removal percentage should be greater than or equal to 50% in an adsorption lifetime test).

The filter cartridge of embodiments 1-4 were examined via the abovementioned tests, the results are illustrated in Table 1.

TABLE 1

| | Specific | Adsorption | Adsorption | Residual |
|---|---|---|---|---|
| | surface area of | value of | value of | chlorine |
| | nanopores | methylene blue | iodine | removal |
| Embodiment | (m²/g) | (mg/g) | (mg/g) | percentage |
| 1 | 2105 | 202 | 1200 | 82.6% |
| 2 | 2060 | 212 | 1300 | 83.2% |
| 3 | 2030 | 210 | 1260 | 81.8% |
| 4 | 2100 | 208 | 1260 | 82.7% |

From the results in Table 1, it can be concluded that embodiments 1-4 of the present invention has high nano-porosity, strong adsorption abilities, and long adsorption lifetime.

The above embodiments are merely the preferred embodiments of the present invention and are not intended to limit the scope of the present invention. Any insubstantial changes and substitutions made by those skilled in the art based on the present invention are within the scope of the present invention.

What is claimed is:

1. A filter cartridge, wherein it includes a filter cartridge body, the filter cartridge body comprises a plastic skeleton and a flat membrane which coats an outer surface of the plastic skeleton; the plastic skeleton is provided with a plurality of water passage holes; a carbon nanofiber composite water purification material is provided on an outer surface of the flat membrane, the carbon nanofiber composite water purification material is prepared by the following components in parts by weight: 70-80 parts of activated carbon cellulose, 6-10 parts of lemon powder, 6-10 parts of Jew's ear powder, and 1-2 parts of graphene oxide.

2. The filter cartridge according to claim 1, wherein the carbon nanofiber composite water purification material binds to the outer surface of the flat membrane by wet centrifugal molding.

3. The filter cartridge according to claim 1, wherein a nonwoven fabric which coats the filter cartridge is provided.

4. The filter cartridge according to claim 1, wherein the plastic skeleton is cylindrically-shaped; the flat membrane is ring-shaped; the flat membrane is coated on the plastic skeleton by welding; the flat membrane has a diameter of 0.1-0.3 μm.
